(19) United States
(12) Patent Application Publication
(10) Pub. No.: US 2022/0017861 A1
(43) Pub. Date: Jan. 20, 2022

(54) UREMIC VASCULOPATHY MODEL USING ENDOTHELIAL CELLS DERIVED FROM INDUCED PLURIPOTENT STEM CELLS AND UREMIC COMPLEX, AND USE THEREOF

(71) Applicant: SAMSUNG LIFE PUBLIC WELFARE FOUNDATION, Seoul (KR)

(72) Inventors: Hye Ryoun JANG, Seoul (KR); Yoon Goo KIM, Seoul (KR); Woo Seong HUH, Seoul (KR); Jae Wook KO, Seoul (KR); Jung Ryul KIM, Seoul (KR)

(21) Appl. No.: 17/275,659

(22) PCT Filed: Sep. 10, 2019
(86) PCT No.: PCT/KR2019/011676

§ 371 Date: Mar. 11, 2021

(30) Foreign Application Priority Data
Sep. 13, 2018 (KR) 10-2018-0109535

(51) Int. Cl. C12N 5/071; G01N 33/50

(52) U.S. Cl. CPC C12N 5/069; C12N 2503/02; C12N 2506/45; G01N 33/5064

(57) ABSTRACT

The present invention relates to a model of uremic vasculopathy and uses of the same. More specifically, the present invention relates to: a medium composition for producing an endothelial cell model of uremic vasculopathy, using a uremic toxin mixture that includes urea and uric acid and may further include indoxyl sulfate, creatinine, or advanced glycation end products (AGEs); a preparation method for an endothelial cell model of uremic vasculopathy, including the step of treating endothelial cells with the uremic toxin mixtures; an endothelial cell model of uremic vasculopathy, prepared by the preparation method; screening and toxicity testing methods, using the model, for agents that inhibit or treat uremic vasculopathy. The uremic toxin mixtures including urea and uric acid of the present invention, merely by using the uremic toxins among other various kinds of uremic toxins, may enable simulating a uremic milieu in endothelial cells conveniently in a manner similar to when using the uremic serum of an actual patient with chronic kidney disease. In addition, the uremic toxin mixture of the present invention, when used for preparing an endothelial cell model of uremic angiopathy, may produce an endothelial cell model capable of reflecting various signs of uremic vasculopathy. Furthermore, by using endothelial cells differentiated from induced pluripotent stem cells when preparing the endothelial cell model of uremic vasculopathy model, it is possible to produce an endothelial cell model of uremic vasculopathy with genetic characteristics reflected therein, which may be beneficially used in customized drug screening for the treatment of patients with uremic vasculopathy, the development of new drugs, toxicity testing, and the like.

### TECHNICAL FIELD

The present invention relates to a model of uremic vasculopathy and uses of same, and more specifically to a medium composition for modeling uremic vasculopathy, using a uremic toxin mixture that includes urea and uric acid and may further include indoxyl sulfate, creatinine, or advanced glycation end products (AGEs); a method for establishing uremic vasculopathy endothelial cell model, including the step of treating endothelial cells derived from induced pluripotent stem cells with the uremic toxin mixture; an endothelial cell model of uremic vasculopathy, prepared by the preparation method; and a method of screening and toxicity testing using the model, for agents that inhibit or treat uremic vasculopathy.

### BACKGROUND ART

Uremia is caused by various uremic substances that accumulate in the blood. This uremia is a complex “addiction” of uremic toxin retention that causes multifactorial problems in which impairments in several metabolic functions are expressed as clinical problems. Uremia causes dysfunction of endothelial cells and affects several organs and organ systems. For example, it includes cardiovascular complications (high blood pressure, pericarditis, and heart failure), peripheral nervous system disorders (multiple neurological disorders), central nervous system disorders (impairment of memory, concentration and intelligence), hematologic abnormalities (anemia, hemorrhagic tendency, clotting), immune dysfunction (immune suppression), digestive disorders (nausea, vomiting) and the like. However, it is still unclear which of these uremic substances are essentially involved in the development and progression of complications due to uremic toxin, and it is unknown whether uremic complications occur only by a single substance or a complex action by these multiple substances.

Uremia can be caused by chronic or acute renal insufficiency. Chronic uremia is caused by irreversible renal injury. Among them, chronic kidney disease refers to persistent structural abnormalities or histological damage of the kidney for 3 months or more or a continuous decrease in kidney function. Chronic kidney disease is classified as five stages according to the degree of renal dysfunction. If not well managed, it will worsen to the last stage, end-stage kidney disease (ESKD), so that renal replacement therapy such as dialysis or kidney transplantation it is finally required. The main causes of chronic kidney disease in Korea are diabetic kidney disease (41%), hypertension (16%), glomerulonephritis (14%) and the like, and other causes include polycystic kidney disease and other kidney and urinary tract diseases. When kidney function deteriorates, uremic symptoms such as tiredness, itchiness, and loss of appetite develop. In ESKD state, symptoms such as shortness of breath, loss of appetite, and vomiting become more severe, and it is impossible to live a normal life without renal replacement therapy such as dialysis or kidney transplantation. Cardiovascular complications are the leading cause of death in patients with chronic kidney disease, especially ESKD. In ESKD patients, uremic toxin causes dysfunction of vascular endothelial cells and uremic vasculopathy in which atherosclerosis progresses very rapidly, facilitating the development of coronary artery disease, peripheral vascular disease, and cerebrovascular disease. Uremic vasculopathy is a fundamental cause of cardiovascular complications and is a major cause of increased mortality and medical expenses in ESKD patients, so that a method that can effectively treat uremic vasculopathy is needed.

For the diagnosis and treatment of chronic kidney disease, blood tests are used to check uremic toxins, electrolyte levels, and acid-base status. In order to prevent the progression of chronic kidney disease and uremic vasculopathy therefrom, it is essential to manage and treat hypertension, diabetes mellitus, and hyperlipidemia as well as treatment according to the primary cause of chronic kidney disease.

As a result of uremia, a number of serious complications occur, therefore effective treatment is needed. It is required to develop a new drug that can effectively treat uremic vasculopathy, which is one of the main factors that increase mortality and medical expenses of patients with chronic kidney disease, especially ESKD, and to develop an effective drug screening method.

### DISCLOSURE

### Technical Problem

Accordingly, while studying to evaluate endothelial dysfunction, which is the basis of uremic vasculopathy, using an induced pluripotent stem cell-derived endothelial cell and a uremic toxin mixture, the inventors of the present invention confirmed that it was possible to create a milieu that induces dysfunction in endothelial cells, similar to when serum of a chronic kidney disease patient with was used directly when mixed with a cell culture solution with different combinations and concentrations of uremic toxin, thereby completing the present invention.

Therefore, the object of the present invention provides a medium composition for producing an endothelial cell model of uremic vasculopathy, including a uremic toxin mixture that includes urea and uric acid and may further include indoxyl sulfate, creatinine, or advanced glycation end products (AGEs), a preparation method for an endothelial cell model of uremic vasculopathy, including the step of treating endothelial cells with uremic toxin mixtures, an endothelial cell model of uremic vasculopathy, prepared by the preparation method, and screening and toxicity testing methods, using the model, for agents that inhibit or treat uremic vasculopathy.

### Technical Solution

In order to achieve the above objective, the present invention provides a medium composition for producing a uremic vasculopathy endothelial cell model, the composition including a uremic toxin mixture including urea and uric acid.

Further, the present invention provides a method for producing a uremic endothelial vasculopathy endothelial cell model, the method including the step of treating an endothelial cell with uremic toxin mixtures including urea and uric acid.

Further, the present invention provides a uremic vasculopathy endothelial cell model produced by the method for producing a uremic vasculopathy endothelial cell model.

Further, the present invention provides a method of screening an agent of inhibiting or treating uremic vasculopathy, the method including the steps of: (S1) treating a uremic vasculopathy endothelial cell model with a candidate substance; (S2) measuring at least one level selected from the group consisting of the level of reactive oxygen production, the level of tube formation, and the level of apoptosis in the uremic vasculopathy endothelial cell model treated with the candidate substance; and (S3) determining the candidate substance as a substance for inhibiting or treating uremic vasculopathy when the production of reactive oxygen species is decreased, the level of tube formation is increased, or the level of apoptosis is decreased compared to the uremic vasculopathy endothelial cell model in which the candidate substance is not treated in the step (S2).

Further, the present invention provides a medium composition for producing a uremic vasculopathy endothelial cell model, the composition including a uremic toxin mixture including urea and uric acid.

Further, the present invention provides a use of a medium composition including uremic toxin mixtures including urea and uric acid for producing a medium composition for producing a uremic vasculopathy endothelial cell model.

### Advantageous Effects

By using the uremic toxin mixtures including urea and uric acid, the uremic toxin alone among various uremic toxins may easily create a uremic milieu in endothelial cells similar to when using serum from an actual patient with chronic kidney disease. Further, by preparing a method for producing a uremic vasculopathy endothelial cell model using the uremic toxin mixture of the present invention, it may prepare an endothelial cell model that may reflect various signs of uremic vasculopathy. When preparing the uremic vasculopathy endothelial cell model, the endothelial cells differentiated from induced pluripotent stem cells are used to manufacture the uremic vasculopathy endothelial cell model that reflects the patient's genetic characteristics so that it may be beneficially used for customized drug screening for the treatment of uremic vasculopathy patients, development of new drugs, and toxicity testing.

### DESCRIPTION OF DRAWINGS

FIG. 1 is a view showing an induced pluripotent stem cell for preparing a uremic vasculopathy endothelial cell model of the present invention.

FIG. 2 is a view showing endothelial cells differentiated from the induced pluripotent stem cells of FIG. 1.

FIG. 3 is a view showing the results of reactive oxygen species produced by endothelial cells in order to analyze the effects of uremic milieu in the body occurring in patients with chronic kidney disease.

FIG. 4 is a view showing the degree of apoptosis of endothelial cells in order to check the effect of uremic milieu in the body occurring in patients with chronic kidney disease.

FIG. 5 is a view showing the results of reactive oxygen species produced by endothelial cells on the 4th day after treatment of the uremic toxin mixture in order to check the effect of the uremic toxin mixture of the present invention on endothelial cells.

FIG. 6 is a view showing the results of reactive oxygen species produced by endothelial cells on the 2nd and 4th days after treatment of the uremic toxin mixture in order to check the effect of the uremic toxin mixture of the present invention on endothelial cells.

FIG. 7 is a view showing the degree of apoptosis of endothelial cells after treatment with the uremic toxin mixture for 4 hours in order to check the effect of the uremic toxin mixture of the present invention on endothelial cells.

FIG. 8 is a view showing the results of tube formation of endothelial cells in the controls 1, 2, 4, and 5, UA 1, and UT 1 groups after treatment with the uremic toxin mixture in order to check the effect of the uremic toxin mixture of the present invention on endothelial cells.

FIG. 9 is a view showing the results of tube formation of endothelial cells in the controls 1 and 3, UA 3, UT E, UT F, and UT H groups after treatment with the uremic toxin mixture in order to check the effect of the uremic toxin mixture of the present invention on endothelial cells.

FIG. 10 is a view showing the effects of the serum of ESKD patients and the uremic toxin mixture of the present invention by measuring the degree of tube formation in order to check the effectiveness and reproducibility of the uremic vasculopathy endothelial cell model prepared by treating endothelial cells with the uremic toxin mixture of the present invention.

FIG. 11 is a view showing the results of tube formation of endothelial cells after treatment with SD-208 and losartan in order to confirm the utility of the uremic vasculopathy endothelial cell model of the present invention as drug screening system.

### BEST MODES OF THE INVENTION

The present invention provides a medium composition for producing a uremic vasculopathy endothelial cell model, the composition including a uremic toxin mixture including urea and uric acid.

The term “uremic toxin mixture” of the present invention refers to a uremic mixture containing urea and uric acid, among various uremic toxins. Further, the uremic toxin mixture of the present invention is complex for simulating the uremic milieu in the body of chronic kidney disease patients and is a uremic mixture capable of inducing uremic angiopathy in endothelial cells.

The uric acid may be included in the uremic toxin mixture at a concentration of 0.55 mM to 1 mM, preferably 0.8 mM (13 mg/dL) to 1 mM (17 mg/dL). When uric acid is included in less than 0.55 mM (9 mg/dL), the amount of reactive oxygen species production of endothelial cells is not increased and the tube formation ability is not affected, and thus in endothelial cells, the uremic milieu in the body that occurs in chronic kidney disease patients is not simulated. Further, when the uric acid may be included in excess of 1 mM, it is extraordinary range of hyperuricemia commonly seen in patients with chronic kidney disease and may cause excessive damage to endothelial cells beyond the uremic milieu occurring in the body of chronic kidney disease patients.

The urea may be included in the uremic toxin mixture at a concentration of 20 mM to 55 mM, preferably 25 mM (71 mg/dL) to 50 mM (143 mg/dL). When the urea is included in the uremic toxin mixture with the above concentration range, it may more effectively implement the uremic milieu occurring in the body of chronic kidney disease patients.

The uremic toxin mixture may further include indoxyl sulfate or advanced glycation end products (AGEs), and the types of uremic toxin that may be included are not limited thereto. The indoxyl sulfate may be included in the uremic toxin mixture at a concentration of 0.3 mM to 1.5 mM, preferably 0.5 mM to 1 mM, and the advanced glycation end products may be included in the uremic toxin mixture at a concentration of 0.5 mg/L to 15 mg/L, preferably 1 mg/L to 10 mg/L. When the indoxyl sulfate or the advanced glycation end products is included in the uremic toxin mixture with the concentration range as described above, it may further increase or maintain the level that appears in the uremic milieu occurring in the body of chronic kidney disease patients that is implemented with the uremic toxin mixture of the present invention including basic components (urea and uric acid).

Further, the uremic toxin mixture may further include creatinine, and the creatinine may be included in the uremic toxin mixture at a concentration of 0.05 mM to 1.5 mM, preferably 0.1 mM (1 mg/dL) to 1 mM (11 mg/dL). When the creatinine is included in the uremic toxin mixture with the above concentration range, it may more effectively implement the body uremic milieu that occurs in chronic kidney disease patients.

The “uremic vasculopathy” is a disease showing a characteristic of rapidly progressive arteriosclerosis caused by endothelial dysfunction due to uremia in chronic kidney disease. Chronic kidney disease, characterized by irreversible renal dysfunction, significantly and progressively impairs the excretion of various uremic toxins, resulting in uremia, thereby inducing endothelial cell dysfunction. Vasculopathy initiated by endothelial dysfunction is a vascular disease caused by damage to endothelial cells and is facilitated by damage to endothelial cells due to uremic toxin; or various uremic toxins that induces endothelial cell damage due to disease caused by the uremic toxin.

The “endothelial cells” of the present invention may be endothelial cells differentiated from induced pluripotent stem cells (iPSCs), but are not limited thereto. In the case of using endothelial cells differentiated from induced pluripotent stem cells as described above, an endothelial cell injury model of uremic vasculopathy reflecting the genetic characteristics may be prepared. The induced pluripotent stem cells may include all induced pluripotent stem cells derived from normal persons or patients with chronic kidney disease. Because endothelial cells derived from induced pluripotent stem cells specific for patients with chronic kidney disease are endothelial cells that reflect characteristics according to uremic vasculopathy severity, and thus, endothelial cells with different characteristics depending on the degree of vascular complications of the patient may be used to prepare uremic vasculopathy endothelial cell models, which is genetically and functionally distinguished. Further, such a model may be beneficially used to screen drugs suitable for the genetic and functional characteristics of each patient and to test the presence or absence of drug toxicity to each patient.

The “medium composition” refers to a medium capable of supporting cell growth and survival ex vivo and in vitro, and includes all conventional media used in the art suitable for culturing cells. The medium composition may include essential and non-essential amino acids, vitamins, energy sources, lipids, trace elements, and the like necessary for minimal proliferation and/or survival of cells.

By using a medium composition for producing uremic vasculopathy endothelial cell model including the uremic toxin mixture including urea and uric acid of the present invention, simple uremic toxin mixture alone among various uremic toxins may effectively establish the uremic vasculopathy endothelial cell model simulating a uremic milieu similar to uremic serum from an actual patient with chronic kidney disease.

Further, the present invention provides a medium composition for producing uremic vasculopathy endothelial cell model, the composition including a uremic toxin mixture including urea and uric acid.

Further, the present invention provides a use of a medium composition including uremic toxin mixture including urea and uric acid for producing medium composition for producing a uremic vasculopathy endothelial cell model.

Further, the present invention provides a method for producing a uremic vasculopathy endothelial cell model, the method including the step of treating an endothelial cell with uremic toxin mixture including urea and uric acid. The endothelial cells may be endothelial cells differentiated from induced pluripotent stem cells (iPSCs), for example, endothelial cells derived from induced pluripotent stem cells specific for patients with chronic kidney disease, but are not limited thereto. The preparation method may be preferably carried out in vitro.

When the uremic vasculopathy endothelial cell model is prepared through the step of treating the endothelial cells with the uremic toxin mixture including urea and uric acid of the present invention, the uremic toxin mixture alone may simply and effectively produce the uremic vasculopathy endothelial cell model having a uremic milieu, similar to uremic serum of an actual patient with chronic kidney disease. Description of each configuration of the preparation method of the present invention is excluded in order to avoid overlapping with the description of the medium composition.

Further, the present invention provides the uremic vasculopathy endothelial cell model produced by the method of producing the uremic vasculopathy endothelial cell model. The endothelial cells may be endothelial cells differentiated from induced pluripotent stem cells (iPSCs), for example, endothelial cells derived from induced pluripotent stem cells specific for patients with chronic kidney disease, but are not limited thereto.

The uremic vasculopathy endothelial cell model prepared by the method for producing a uremic vasculopathy endothelial cell model of the present invention includes a uremic milieu similar to uremic serum from an actual patient with chronic kidney disease, such as a similar level of reactive oxygen production, level of tube formation and level of apoptosis, but is not limited thereto. Therefore, the endothelial cell model of the present invention is a model that simply reflects the signs of uremic vasculopathy using the uremic toxin mixture of the present invention and may be effectively used for drug screening and toxicity testing for the treatment of patients with uremic vasculopathy.

Further, the present invention provides a method for screening an agent of inhibiting or treating uremic vasculopathy, the method including the steps of: (S1) treating a uremic vasculopathy endothelial cell model with a candidate substance; (S2) measuring at least one level selected from the group consisting of the level of reactive oxygen species, the level of tube formation, and the level of apoptosis in the uremic vasculopathy endothelial cell model treated with the candidate substance; and (S3) determining the candidate substance as a substance for inhibiting or treating uremic vasculopathy when the production of reactive oxygen species is decreased, the level of tube formation is increased, or the level of apoptosis is decreased compared to the endothelial cells of uremic vasculopathy endothelial cell model in which the candidate substance is not treated in the step (S2).

The step (S1) is a step of treating a candidate substance to a uremic vasculopathy endothelial cell model in which the uremic milieu is simulated by the uremic toxin mixture of the present invention. In step (S1), the “candidate substance” is a substance expected to treat, prevent or ameliorate uremic vasculopathy induced by endothelial cell dysfunction in chronic kidney disease and includes chemical substances, oligonucleotides, peptides, genes, proteins, foods, formulations, compounds, compositions, medicines, nutritional supplements, health care products or beverages, without limitation. Further, the “treatment” may be a method of including a candidate substance in a medium composition for preparing a uremic vasculopathy endothelial cell model, but is not limited thereto.

The step (S2) is a step of measuring various level changes to determine whether the uremic milieu of the uremic vasculopathy endothelial cell model treated with the candidate substance is improved and may measure one or more levels selected from the group consisting of the level of reactive oxygen species, the level of tube formation and the level of apoptosis, but is not limited thereto. The “measurement” may be performed by conventional methods known in the art.

In step (S3), as a result of the measurement in step (S2), the candidate substance selected by improvement of the uremic milieu in uremic vasculopathy endothelial cell model is determined as a substance for inhibiting or treating uremic vasculopathy. In step (S3), the candidate substance may be determined as a substance for inhibiting or treating uremic vasculopathy when the production of reactive oxygen species is decreased, when the level of tube formation is increased, or when the level of apoptosis is decreased compared to the uremic vasculopathy endothelial cell model in which the candidate substance is not treated in step (S2).

The endothelial cells may be endothelial cells differentiated from induced pluripotent stem cells (iPSCs), and may be endothelial cells derived from normal induced pluripotent stem cells or induced pluripotent stem cells specific for patients with chronic kidney disease, but are not limited thereto. The endothelial cells differentiated from induced pluripotent stem cells are used so that the uremic vasculopathy endothelial cell model reflecting genetic characteristics may be used to screen an agent for inhibiting or treating the uremic vasculopathy. In particular, the uremic vasculopathy endothelial cell model is used so that the endothelial cells differentiated from the patient-specific induced pluripotent stem cells may be used to screen a substance inhibiting or treating uremic vasculopathy, which exhibits an effect according to the characteristics and degree of vascular complications of the patient.

Hereinafter, the present invention will be described in more detail based on Examples. The Examples are only for describing the present invention in more detail, and it will be apparent to those of ordinary skill in the art that the scope of the invention according to the gist of the present invention is not limited by these Examples.

### Modes of the Invention

### Example 1. Preparation of Uremic Vasculopathy Endothelial Cell Model

### Example 1-1. Endothelial Cell Differentiation

In order to prepare the uremic vasculopathy endothelial cell model, first, the induced pluripotent stem cells from the normal control were differentiated by a method verified in the existing documents (Gimbrone et al. Circ Res. 2016; 118:620-636/Chen et al. Nat Rev Cardiol. 2016; 13: 333-349/Paik et al. Circ Res. 2018 Jul. 9.Epub ahead of print).

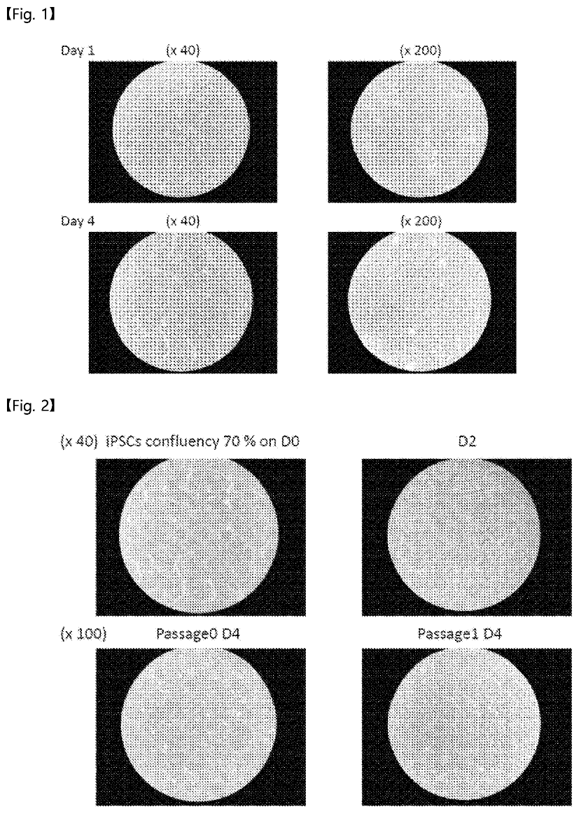

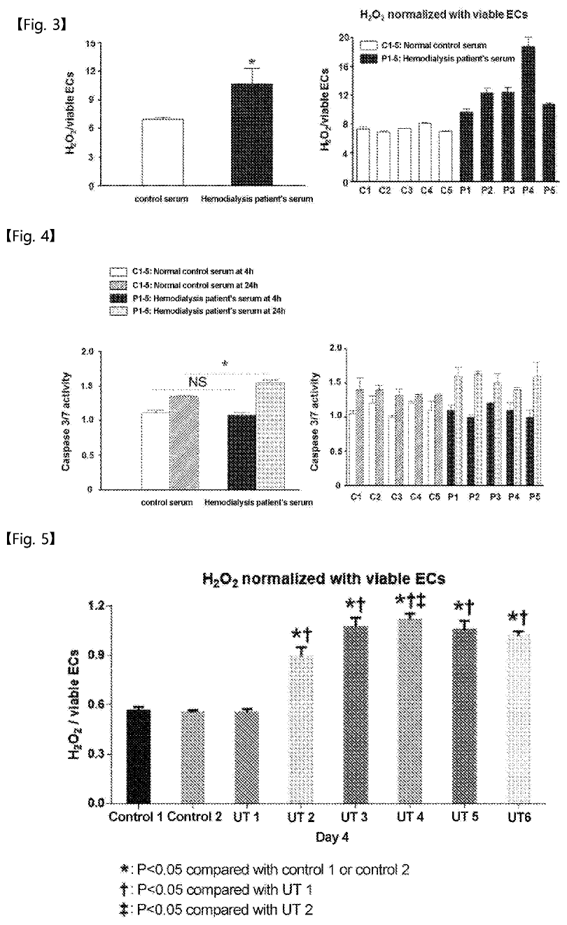

FIG. 1 shows the induced pluripotent stem cells from the normal control, and FIG. 2 shows the endothelial cells differentiated from the induced pluripotent stem cells of the normal control.

### Example 1-2. Determination of Type and Composition of Uremic Toxin Mixture

In order to prepare the uremic vasculopathy endothelial cell model, it is necessary to implement a uremic milieu occurring in the body of chronic kidney disease patient. In order to experimentally implement this milieu, considering the clinical significance, several combinations of uremic toxin mixtures were applied to differentiated endothelial cells, and the effects of each combination of uremic toxin mixtures on endothelial cells were investigated. The list and composition of uremic toxin and uremic toxin mixtures used in the experiment for identifying an appropriate mixture of uremic toxins to simulate uremic milieu are shown in Tables 1 and 2. The control group 1 using only media was commonly included in the experiment conducted with all the control and experimental groups in Table 2.

TABLE 1

| Uremic toxin | Conventional unit | | SI unit | |
|---|---|---|---|---|
| Urea nitrogen | 14.01 | mg/dL | 5 | mmol/L |
| | 70.01 | mg/dL | 25 | mmol/L |
| | 140.06 | mg/dL | 50 | mmol/L |
| | 210.08 | mg/dL | 75 | mmol/L |
| Creatinine (Cr) | 1.13 | mg/dL | 0.1 | mmol/L |
| | 11.3 | mg/dL | 1 | mmol/L |
| Uric acid (UA) | 4.2025 | mg/dL | 0.25 | mmol/L |
| | 8.405 | mg/dL | 0.5 | mmol/L |
| | 13.45 | mg/dL | 0.8 | mmol/L |
| | 16.81 | mg/dL | 1 | mmol/L |

| | | |
|---|---|---|
| Indoxyl sulfate (IS) | 0.5 | mmol/L |

| | |
|---|---|
| Advanced glycation end products | 1 mg/L, 10 mg/L |
| (AGE) | |

TABLE 2

| | | | | | | Advanced | |
|---|---|---|---|---|---|---|---|
| | | | | | | glycation | |
| | | | | | Indoxyl | end | |
| | | Urea | Creatinine | Uric acid | sulfate | products | NaOH |
| No | Name | (mM) | (mM) | (mM) | (mM) | (mg/L) | (μL/mL) |
| 1 | Control 1 | | | | | | |
| | (media only) | | | | | | |
| 2 | Control 2 | 5 | 0.1 | | | | |
| | (normal | | | | | | |
| | physiologic | | | | | | |
| | levels) | | | | | | |
| 3 | Control 3 | 5 | 0.1 | 0.25 | | | 11 |
| 4 | Control 4 | 5 | 0.1 | 0.25 | | | |
| 5 | Control 5 | | | | | | 16 |
| 6 | UA 1 | | | 0.5 | | | |
| 7 | UA 2 | | | 1 | | | |
| 8 | UA 3 | 5 | 0.1 | 0.8 | | | |
| 9 | Urea | 50 | | | | | |
| 10 | Cr | | 1 | | | | |
| 11 | IS | | | 1 | | | |
| 12 | Uremic toxin | 50 | 1 | | | | |
| | mixture 1 | | | | | | |
| | (UT 1) | | | | | | |
| 13 | Uremic toxin | 50 | 1 | 1 | | | |
| | mixture 2 | | | | | | |
| | (UT 2) | | | | | | |
| 14 | Uremic toxin | 50 | 1 | 1 | 0.5 | | |
| | mixture 3 | | | | | | |
| | (UT 3) | | | | | | |
| 15 | Uremic toxin | 50 | 1 | 1 | 0.5 | 1 | |
| | mixture 4 | | | | | | |
| | (UT 4) | | | | | | |
| 16 | Uremic toxin | 25 | 1 | 1 | 0.5 | 10 | |
| | mixture 5 | | | | | | |
| | (UT 5) | | | | | | |
| 17 | Uremic toxin | 50 | 1 | 1 | 0.5 | 10 | |
| | mixture 6 | | | | | | |
| | (UT 6) | | | | | | |
| 18 | Uremic toxin | 25 | 1 | 0.5 | | | |
| | mixture A | | | | | | |
| | (UT A) | | | | | | |
| 19 | Uremic toxin | 25 | 1 | 1 | | | |
| | mixture B | | | | | | |
| | (UT B) | | | | | | |
| 20 | Uremic toxin | 50 | 1 | 0.5 | | | |
| | mixture C | | | | | | |
| | (UT C) | | | | | | |
| 21 | Uremic toxin | 50 | 1 | 1 | | | |
| | mixture D | | | | | | |
| | (UT D) | | | | | | |
| 22 | Uremic toxin | 25 | 1 | 0.8 | | | |
| | mixture E | | | | | | |
| | (UT E) | | | | | | |
| 23 | Uremic toxin | 25 | 1 | 0.8 | 1 | | |
| | mixture F | | | | | | |
| | (UT F) | | | | | | |
| 24 | Uremic toxin | 50 | 1 | 0.8 | | | |
| | mixture G | | | | | | |
| | (UT G) | | | | | | |
| 25 | Uremic toxin | 50 | 1 | 0.8 | 1 | | |
| | mixture H | | | | | | |
| | (UT H) | | | | | | |

### Comparative Example 1. Measurement of Uremic Milieu Occurring in Body of Chronic Kidney Disease Patients

Before confirming the effectiveness of the uremic vasculopathy endothelial cell model treated with the uremic toxin mixture of the present invention, the endothelial cell model of the present invention was treated with the serum of an ESKD patient undergoing actual hemodialysis, confirming the result of measuring the level of a uremic milieu occurring in the body of chronic kidney disease patients, so as to compare the effect with the uremic toxin mixture of the present invention. Serum from patients with ESKD was used as serum from patients undergoing hemodialysis at Samsung Medical Center (IRB file number 2016-11-025).

### Comparative Example 1-1. Measurement of Level of Reactive Oxygen Species (ROS) Production of Endothelial Cells

After treating the patient's serum in the endothelial cell model of the present invention, the level of reactive oxygen species produced by the endothelial cells was measured according to the manufactural instructions using the ROS-Glo™ H₂O₂ assay kit (Promega, catalog number G8820), and the results are shown in FIG. 3.

As shown in FIG. 3, uremic serum of hemodialysis patients significantly increased the production of reactive oxygen species from endothelial cells compared to normal serum.

### Comparative Example 1-2. Measurement of Apoptosis of Endothelial Cell

After treating the patient's serum in the endothelial cell model of the present invention, apoptosis of endothelial cell was measured according to the manufactural instructions using the Caspase-Glo 3/7 assay systems kit (Promega, catalog number G8091), and the results are shown in FIG. 4.

As shown in FIG. 4, apoptosis was significantly promoted in endothelial cells treated with the patient's serum for 24 hours.

Therefore, the result of the Examples confirmed that the level of reactive oxygen species and the level of apoptosis were increased when the serum of a patient with chronic kidney disease was actually treated in the endothelial cell model.

### Example 2. Confirmation of the Effect of Uremic Toxin Mixture on Endothelial Cells and Confirmation of Optimal Uremic Toxin Mixture Composition

### Example 2-1. Measurement of the Production of Reactive Oxygen Species

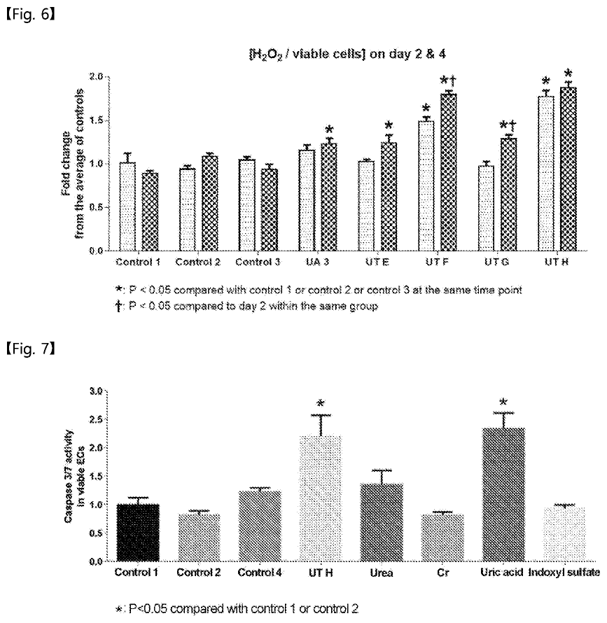

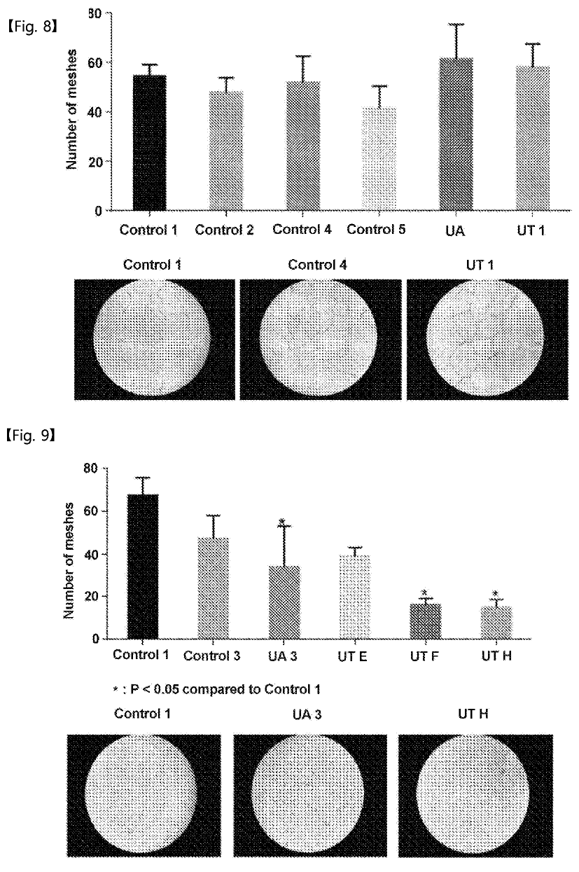

In order to confirm whether the uremic milieu in the body was simulated as when the actual patient's serum was applied in Comparative Example 1 in the endothelial cell model to which the uremic toxin alone substance or the uremic toxin mixture was applied, as shown in Table 2, the level of reactive oxygen production was measured. The results are shown in FIGS. 5 and 6. The results for the 4th day of application of the uremic toxin mixture are shown in FIG. 5, and the results for the 2nd and 4th days of the application are shown in FIG. 6.

As shown in FIG. 5, it was confirmed that compared with Control 1 to which only a general medium was added and Control 2 to which urea and creatinine were added at a normal physiological concentration, no change was observed even when urea and creatinine were treated at high concentrations (UT 1), and that the level of reactive oxygen species was rapidly increased when mixed and added with uric acid (UT 2 to UT 6). Further, referring to the results from UT2 to UT6, even if the uremic toxin mixture with the addition of indoxyl sulfate and the advanced glycation end product were treated (UT 3 to UT 6), its level of reactive oxygen species was increased similar to the experimental group without these additions (UT 2).

Therefore, it was confirmed that urea, creatinine, and uric acid are required as basic components in the uremic toxin mixture for a significant increase in reactive oxygen species. Further, it was confirmed that adding indoxyl sulfate or advanced glycation end product could further increase the production of reactive oxygen species or maintain a similar level of production.

Further, as shown in FIG. 6, it was confirmed that a significant difference appeared in the experimental group containing the uric acid at a concentration of 0.8 mM or more (UA 3) compared to the Control 3 including urea, creatinine and uric acid, which are the basic components of the uremic toxin mixture of the present invention. Further, referring to the results of the UT F and UT H experimental groups, it was confirmed that the production of reactive oxygen species increased even when the addition of indoxyl sulfate to basic components of urea, creatinine and uric acid.

### Example 2-2. Confirmation of Apoptosis Promotion

Further, as in the above Example, in order to confirm whether the uremic milieu in the body was simulated as when the actual patient's serum was applied in Comparative Example 1 in the endothelial cell model to which the uremic toxin alone substance or the uremic toxin mixture was applied as shown in Table 2, the level of apoptosis was measured. The results are shown in FIG. 7.

As shown in FIG. 7, it was confirmed that apoptosis was significantly promoted in endothelial cells treated with the uremic toxin mixture (urea, creatinine, uric acid, indoxyl sulfate) (UT H) of the present invention for 4 hours. Meanwhile, referring to the results in the group treated with the uremic toxin mixture alone, it was confirmed that apoptosis was not significantly promoted even when treated with urea (50 mM), creatinine (1 mM), and indoxyl sulfate (1 mM). It was confirmed that only for uric acid, apoptosis was significantly promoted in endothelial cells only when added at 1 mM.

### Example 2-3. Measurement of Tube Formation Ability

Further, in order to confirm whether the uremic milieu in the body was simulated in the same way as when the actual patient's serum was applied in the endothelial cell model to which the uremic toxin alone substance or the uremic toxin mixture was applied, the level of tube formation was measured and compared as follows.

In order to evaluate the functional abnormality of endothelial cells that may form vascular structures, the tube formation ability was measured. After seeding endothelial cells into a 24 or 12-well plate applied with Matrigel (Corning, catalog number CB-40234). After 16 to 24 hours of incubation, the level of tube formation was compared and analyzed. The results are shown in FIGS. 8 to 10.

As shown in FIG. 8, it was confirmed that the tube formation ability of endothelial cells was well maintained in the experimental groups (control 1, 2, 4, 5, UA 1, UT 1) that did not contain uric acid or did not contain 0.8 mM or more of uric acid.

Further, as shown in FIG. 9, it was confirmed that the level of tube formation was suppressed in the experimental groups (UA 3, UT E) containing 0.8 mM or more of uric acid, and the tube formation was significantly suppressed in the experimental groups (UT F, UT H) further containing indoxyl sulfate.

Therefore, referring to the above three-level measurement results, it was confirmed that the uremic toxin mixture showing significant results in common at various measurement levels to confirm whether the uremic milieu in the body is simulated as when the actual patient's serum is applied was the uremic toxin mixture including the urea and uric acid as essential components.

### Example 3. Confirmation of Effectiveness of Uremic Vasculopathy Endothelial Cell Model

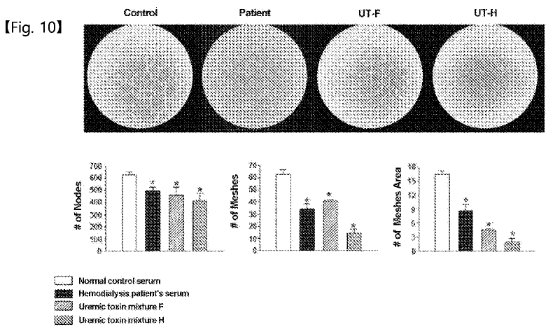

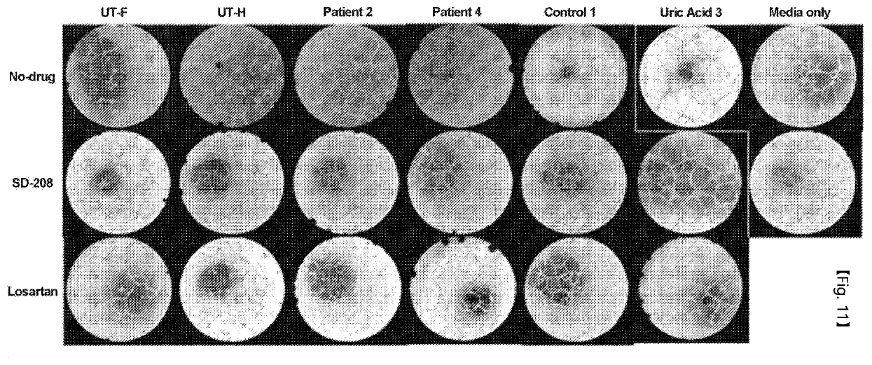

In order to confirm the reliability and effectiveness of the uremic vasculopathy endothelial cell model prepared by treating endothelial cells with the uremic toxin mixture of the present invention, the level of tube formation was measured to compare effects of the serum of ESKD patient undergoing hemodialysis and the uremic toxin mixture of the present invention by measuring the tube formation level. The results are shown in FIG. 10.

As shown in FIG. 10, it was confirmed that the uremic toxin mixture of the present invention (UT F, UT H) significantly inhibited the tube formation ability of endothelial cells, similar to the effect of actual patient's serum.

Therefore, it was confirmed that the uremic toxin mixture of the present invention is effective in preparing a uremic vasculopathy endothelial cell model.

### Example 4. Experiment of Verification of Drug Effect Using Uremic Vasculopathy Endothelial Cell Model

In order to confirm whether the effect of the drug can be verified using the uremic vasculopathy endothelial cell model prepared by treating with the uremic toxin mixture of the present invention, the uremic toxin mixture of the present invention or ESKD patient's serum was applied to endothelial cells derived from normal control induced pluripotent stem cells. Then, SD-208 (TGF-β inhibitor) and losartan (angiotensin receptor blocker) were treated to measure the level of tube formation in order to examine respective effects. The results are shown in FIG. 11. The endothelial cells' tube formation ability was measured and compared in the uremic toxin mixture of the present invention (UT-F, UT-H, uric acid 3 (UA 3)), serum of patients with chronic kidney disease (patient 2, patient 4), normal control serum (control 1) and cell culture medium alone (media only). The results are as follows.

As shown in FIG. 11, it was confirmed that the tube formation ability of endothelial cells was well maintained in the normal control serum and the cell culture medium only, and the tube formation was inhibited in both group of the uremic toxin mixture of the present invention and the serum of patients with chronic kidney disease. Further, it was confirmed that inhibition of endothelial cell tube formation was alleviated in the group of the uremic toxin mixture and the serum of ESKD patients treated with SD-208 and losartan.

Therefore, the treatment with a uremic toxin mixture composed of uremic toxins selected in the present invention may produce the endothelial cell model reflecting the signs of a uremic vasculopathy and induce results similar to those using the actual ESKD patient's serum, thereby confirming that the uremic vasculopathy endothelial cell model prepared by treating with the uremic toxin mixture of the present invention can be used for drug screening in patients with uremic vasculopathy.

1. A medium composition for producing a uremic vasculopathy endothelial cell model, the composition comprising uremic toxin mixtures including urea and uric acid.

2. The medium composition for producing a uremic vasculopathy endothelial cell model of claim 1, wherein the uric acid is included at a concentration of 0.55 mM to 1 mM in the uremic toxin mixtures.

3. The medium composition for producing a uremic vasculopathy endothelial cell model of claim 1, wherein the urea is included at a concentration of 20 mM to 55 mM in the uremic toxin mixtures.

4. The medium composition for producing a uremic vasculopathy endothelial cell model of claim 1, wherein the uremic toxin mixture further includes indoxyl sulfate or advanced glycation end product (AGE).

5. The medium composition for producing a uremic vasculopathy endothelial cell model of claim 4, wherein the indoxyl sulfate is included at a concentration of 0.3 mM to 1.5 mM and the advanced glycation end product is included at a concentration of 0.5 mg/L to 15 mg/L in the uremic toxin mixtures.

6. The medium composition for producing a uremic vasculopathy endothelial cell model of claim 1, wherein the uremic toxin mixture further includes creatinine.

7. The medium composition for producing a uremic vasculopathy endothelial cell model of claim 1, wherein the uremic vasculopathy is caused by endothelial cell dysfunction due to chronic kidney disease.

8. A method for producing a uremic vasculopathy endothelial cell model, the method comprising a step of treating an endothelial cell with a uremic toxin mixture including urea and uric acid.

9. The method for producing a uremic vasculopathy endothelial cell model of claim 8, wherein the endothelial cells are derived from induced pluripotent stem cells (iPSCs).

10. A uremic vasculopathy endothelial cell model produced by the method for producing a uremic vasculopathy endothelial cell model of claim 8.

11. The uremic vasculopathy endothelial cell model of claim 10, wherein the endothelial cells are derived from induced pluripotent stem cells (iPSCs).

12. A method of screening an agent of inhibiting or treating uremic vasculopathy, the method comprising steps of:
(S1) treating a uremic vasculopathy endothelial cell model of claim 8 with a candidate substance;
(S2) measuring at least one level selected from the group consisting of a level of reactive oxygen species, a level of tube formation, and a level of apoptosis in the uremic vasculopathy endothelial cell model treated with the candidate substance; and
(S3) determining the candidate substance as a substance for inhibiting or treating the uremic vasculopathy when the production of reactive oxygen species is decreased, the level of tube formation is increased, or the level of apoptosis is decreased compared to the uremic vasculopathy endothelial cell model in which the candidate substance is not treated in the step (S2).

13. The method of screening an agent of inhibiting or treating uremic vasculopathy of claim 12, wherein the endothelial cells are derived from induced pluripotent stem cells (iPSCs).
